(19) United States
(12) Patent Application Publication
(10) Pub. No.: US 2003/0092733 A1
(43) Pub. Date: May 15, 2003

(54) Process for preparing trans-2,4-disubstituted piperidines

(76) Inventors: Marvin Martin Hansen, Indianapolis, IN (US); Perry Clark Heath, Indianapolis, IN (US); Sandra Sabol Keast, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/148,788

(22) PCT Filed: Dec. 6, 2000
(86) PCT No.: PCT/US00/32428

(51) Int. Cl. C07D 211/08; C 07D 4 1/02; A61K 31/445

(52) U.S. Cl. 514/326; 546/207; 546/184; 546/192

(57) ABSTRACT

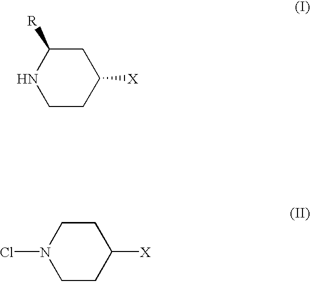

The present invention provides a process for preparing a compound of formula (I), wherein R represents C₁-C₄ alkyl; and X represents an alkyl, alkenyl, cycloalkyl, aryl, substituted aryl, heterocycle, or substituted heterocycle, comprising treating a compound of formula (II), with a suitable crown ether and a suitable base followed by addition of a compound of formula R⁻M⁺ wherein M⁺ is a suitable cation.

The present invention allows for stereoselectively preparing trans-2,4-disubstituted piperidines in high yield. Thus, the present invention provides an efficient synthesis of various trans-2,4-disubstituted piperidines which are useful intermediates in the preparation of pharmaceutical compounds.

The present invention provides a process for preparing a compound of formula I:

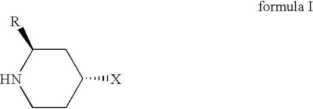

wherein

R represents C₁-C₄ alkyl; and

X represents an alkyl, alkenyl, cycloalkyl, aryl, substituted aryl, heterocycle, or substituted heterocycle, comprising treating a compound of formula II:

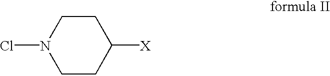

with a suitable base followed by addition of a compound of formula R⁻M⁺ wherein M⁺ is a suitable cation.

The present invention further provides a process for preparing a compound of formula I:

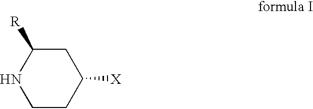

wherein

R represents C₁-C₄ alkyl; and

X represents an alkyl, alkenyl, cycloalkyl, aryl, substituted aryl, heterocycle, or substituted heterocycle, comprising treating a compound of formula II:

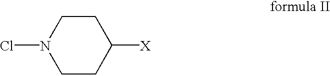

with a suitable crown ether and a suitable base followed by addition of a compound of formula R⁻M⁺ wherein M⁺ is a suitable cation.

In addition, the present invention comprises purifying the compound of formula I by treatment with a suitable reducing agent followed by addition of a suitable acylating agent and then acid-base extraction of the mixture.

Furthermore, the present invention provides novel compounds of formula I:

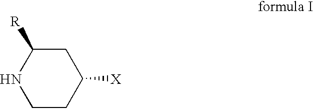

wherein

R represents C₁-C₄ alkyl; and

X represents an alkyl, alkenyl, cycloalkyl, aryl, substituted aryl, heterocycle, or substituted heterocycle.

As used herein, the terms “Me”, “Et”, “Pr”, “iPr”, “Bu” and “t-Bu” refer to methyl, ethyl, propyl, isopropyl, butyl and tert-butyl, respectively.

As used herein, the terms “Halo”, “Halide” or “Hal” refer to a chlorine, bromine, iodine or fluorine atom, unless otherwise specified herein.

As used herein the term “alkyl” refers to a straight or branched, monovalent, saturated aliphatic chain. It is understood that the term “alkyl” includes within its definition the terms “C₁-C₂₀ alkyl”, “C₁-C₁₀ alkyl”, “C₁-C₆ alkyl”, and “C₁-C₄ alkyl”.

As used herein the term “C₁-C₄ alkyl” refers to a straight or branched, monovalent, saturated aliphatic chain of 1 to 4 carbon atoms and includes, but is not limited to methyl, ethyl, n-propyl, isopropyl, n-butyl, isobutyl and the like.

As used herein the term “C₁-C₆ alkyl” refers to a straight or branched, monovalent, saturated aliphatic chain of 1 to 6 carbon atoms and includes, but is not limited to methyl, ethyl, n-propyl, isopropyl, n-butyl, isobutyl, t-butyl, n-pentyl, n-hexyl, and the like.

As used herein the term “C₁-C₁₀ alkyl” refers to a straight or branched, monovalent, saturated aliphatic chain of 1 to 10 carbon atoms and includes, but is not limited to methyl, ethyl, propyl, isopropyl, n-butyl, isobutyl, tertiary butyl, pentyl, isopentyl, hexyl, 2,3-dimethyl-2-butyl, heptyl, 2,2-dimethyl-3-pentyl, 2-methyl-2-hexyl, octyl, 4-methyl-3-heptyl and the like.

As used herein the term “C₁-C₂₀ alkyl” refers to a straight or branched, monovalent, saturated aliphatic chain of 1 to 20 carbon atoms and includes, but is not limited to, methyl, ethyl, propyl, isopropyl, butyl, isobutyl, t-butyl, pentyl, isopentyl, hexyl, 3-methylpentyl, 2-ethylbutyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-nonadecyl, n-eicosyl and the like.

As used herein the term “C₁-C₆ alkoxy” refers to a straight or branched alkyl chain having from one to six carbon atoms attached to an oxygen atom. Typical C₁-C₆ alkoxy groups include methoxy, ethoxy, propoxy, isopropoxy, butoxy, t-butoxy, pentoxy and the like. The term “C₁-C₆ alkoxy” includes within its definition the term “C₁-C₄ alkoxy”.

As used herein the term “halo(C₁-C₆)alkyl” refers to a straight or branched alkyl chain having from one to six carbon atoms with 1, 2 or 3 halogen atoms attached to it. Typical halo(C₁-C₆)alkyl groups include chloromethyl, 2-bromoethyl, 1-chloroisopropyl, 3-fluoropropyl, 2,3-dibromobutyl, 3-chloroisobutyl, iodo-t-butyl, trifluoromethyl and the like. The term “halo(C₁-C₆)alkyl” includes within its definition the term “halo(C₁-C₄)alkyl”.

As used herein the term “cycloalkyl” refers to a saturated hydrocarbon ring structure. It is understood that the term “cycloalkyl” includes within its definition the term “C₃-C₈ cycloalkyl”. Typical C₃-C₈ cycloalkyl groups include cyclopropyl, cyclobutyl, cyclopentyl, cyclohexyl, cycloheptyl, cyclooctyl, and the like.

As used herein the term “alkenyl” refers to a straight or branched, monovalent, unsaturated aliphatic chain. It is understood that the term “alkenyl” includes within its definition the term “C₂-C₆ alkenyl”. Typical C₂-C₆ alkenyl groups include ethenyl (also known as vinyl), 1-methylethenyl, 1-methyl-1-propenyl, 1-butenyl, 1-hexenyl, 2-methyl-2-propenyl, 1-propenyl, 2-propenyl, 2-butenyl, 2-pentenyl, and the like.

As used herein the term “aryl” refers to a monovalent carbocyclic group containing one or more fused or non-fused phenyl rings and includes, for example, phenyl, 1- or 2-naphthyl, 1,2-dihydronaphthyl, 1,2,3,4-tetrahydronaphthyl, and the like.

As used herein the term “heterocycle” refers to a stable 5- to 7-membered monocyclic or 7- to 10-membered bicyclic heterocyclic ring which is saturated or unsaturated, and consists of carbon atoms and from one to three heteroatoms selected from the group consisting of nitrogen, oxygen or sulfur, and wherein the nitrogen and sulfur heteroatoms may optionally be oxidized, and the nitrogen heteroatom may optionally be quaternized and including a bicyclic group in which any of the above-defined heterocyclic rings is fused to a benzene ring. The heterocyclic ring may be attached at any heteroatom or carbon atom which affords a stable structure.

Examples of such heterocycles include piperidinyl, piperazinyl, azepinyl, pyrrolyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazolyl, benzopyranyl, benzothiazolyl, benzoazolyl, furyl, tetrahydrofuryl, tetrahydropyranyl, thienyl, benzothienyl, thiamorpholinyl, thiamorpholinyl-sulfoxide, thiamorpholinylsulfone, oxadiazolyl, triazolyl, tetrahydroquinolinyl, tetrahydrisoquinolinyl, and the like.

The term “substituted” as used in the term “substituted aryl” and “substituted heterocycle” signifies that one or more (for example one or two) substituents may be present on the aryl or heterocycle. Examples of substituents which may be present are H, F, Cl, Br, I, C₁-C₆ alkyl, C₁-C₆ alkoxy, halo(C₁-C₆)alkyl, phenyl, NO₂, NH₂, CN, or phenyl substituted with from 1 to 3 substituents selected from the group consisting of F, Cl, Br, I, C₁-C₆ alkyl, C₁-C₆ alkoxy, halo(C₁-C₆)alkyl, phenyl, NO₂, NH₂, and CN.

The designation “ [symbol] ” refers to a bond that protrudes forward out of the plane of the page.

The designation “ [symbol] ” refers to a bond that protrudes backward out of the plane of the page.

As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. The term “chiral center” refers to a carbon atom to which four different groups are attached. As used herein, the term “diastereomers” refers to stereoisomers which are not enantiomers. In addition, two diastereomers which have a different configuration at only one chiral center are referred to herein as “epimers”. The terms “racemate”, “racemic mixture” or “racemic modification” refer to a mixture of equal parts of enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

$$ee = \frac{{E}^{1} - {E}^{2}}{{E}^{1} + {E}^{2}} \times 100$$

wherein E¹ is the amount of the first enantiomer and E² is the amount of the second enantiomer. Thus, if the initial ratio of the two enantiomers is 50:50, such as is present in a racemic mixture, and an enantiomeric enrichment sufficient to produce a final ratio of 50:30 is achieved, the ee with respect to the first enantiomer is 25%. However, if the final ratio is 90:10, the ee with respect to the first enantiomer is 80%. An ee of greater than 90% is preferred, an ee of greater than 95% is most preferred and an ee of greater than 99% is most especially preferred. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art. In addition, the enantiomers of compounds of formulas I or Ia can be resolved by one of ordinary skill in the art using standard techniques well known in the art, such as those described by J. Jacques, et al., “Enantiomers, Racemates, and Resolutions”, John Wiley and Sons, Inc., 1981.

Some of the compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

The terms “R” and “S” are used herein as commonly used in organic chemistry to denote specific configuration of a chiral center. The term “R” (rectus) refers to that configuration of a chiral center with a clockwise relationship of group priorities (highest to second lowest) when viewed along the bond toward the lowest priority group. The term “S” (sinister) refers to that configuration of a chiral center with a counterclockwise relationship of group priorities (highest to second lowest) when viewed along the bond toward the lowest priority group. The priority of groups is based upon their atomic number (in order of decreasing atomic number). A partial list of priorities and a discussion of stereochemistry is contained in “Nomenclature of Organic Compounds: Principles and Practice”, (J. H. Fletcher, et al., eds., 1974) at pages 103-120.

The specific stereoisomers and enantiomers of compounds of formula (I) can be prepared by one of ordinary skill in the art utilizing well known techniques and processes, such as those disclosed by Eliel and Wilen, “Stereochemistry of Organic Compounds”, John Wiley & Sons, Inc., 1994, Chapter 7, Separation of Stereoisomers. Resolution. Racemization, and by Collet and Wilen, “Enantiomers, Racemates, and Resolutions”, John Wiley & Sons, Inc., 1981. For example, the specific stereoisomers and enantiomers can be prepared by stereospecific syntheses using enantiomerically and geometrically pure, or enantiomerically or geometrically enriched starting materials. In addition, the specific stereoisomers and enantiomers can be resolved and recovered by techniques such as chromatography on chiral stationary phases, enzymatic resolution or fractional recrystallization of addition salts formed by reagents used for that purpose.

The compounds of formula I can be prepared by following the procedures as set forth in Scheme I. All substituents, unless otherwise indicated, are previously defined. The reagents and starting materials are readily available to one of ordinary skill in the art.

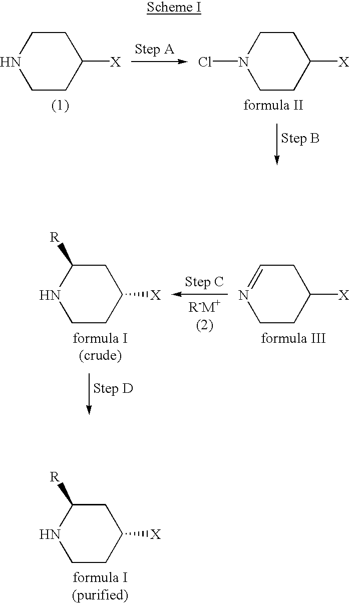

In Scheme I, step A, the compound of structure (1) is treated with a suitable N-chlorinating reagent to provide the compound of formula II. Examples of suitable N-chlorinating reagents are N-chlorosuccinimide, sodium hypochlorite, t-butylhypochlorite, N-chlorophthalimide, and the like. N-chlorosuccinimide is the preferred N-chlorinating reagent. For example, compound (1) is dissolved in a suitable organic solvent, such as diethyl ether and tetrahydrofuran and treated with about 1 equivalent of N-chlorosuccinimide. The reaction mixture is stirred at room temperature for about 30 minutes to 16 hours and the product, compound of formula II, is then isolated by standard techniques well known in the art, such as extraction techniques. For example, the reaction is diluted with saturated aqueous sodium bicarbonate and water, and then extracted with diethyl ether or methyl tert-butyl ether. The organic extracts are combined, dried over anhydrous sodium sulfate, filtered, and concentrated under vacuum to provide the compound of formula II.

In Scheme I, step B, the compound of formula II is dehydrohalogenated with a suitable base in the presence of a suitable crown ether to provide the imine of formula III. Examples of suitable bases are potassium hydroxide, potassium superoxide, and the like. Potassium hydroxide is the preferred suitable base. Examples of suitable crown ethers are 18-crown-6, dibenzo-18-crown-6, and the like. 18-crown-6 is the preferred crown ether. In addition, in Scheme I, step B, the compound of formula II can be dehydrohalogenated with a suitable base which does not require addition of a suitable crown ether, to provide the imine of formula III. Examples of such suitable bases which do not require a suitable crown ether include aqueous sodium hydroxide, Amberlyst® A-27 in THF with no water present, potassium tert-butoxide, lithium tert-butoxide, lithium diisopropylamide (LDA), 1,8-diazabicyclo[5.4.0]undec-7-ene (DBU), and the like.

For example, the compound of formula II is dissolved in a suitable organic solvent, such as tetrahydrofuran and treated with about 0.05 equivalents to about 1.0 equivalents of a crown ether, such as 18-crown-6, with about 0.073 equivalents of crown ether being preferred. The solution is then treated with about 2 equivalents to about 3 equivalents of a suitable base in water, such as potassium hydroxide, with 3 equivalents of suitable base being preferred. The reaction mixture is stirred at room temperature for about 8 to 24 hours and the resulting imine of formula III is isolated by techniques well known in the art, such as drying over anhydrous sodium sulfate and filtering to provide the imine of formula III in solution.

Alternatively, in Scheme I, step B, the compound of formula II is dissolved in a suitable organic solvent, such as tetrahydrofuran and treated with about 2 equivalents to about 3 equivalents of a suitable base, such as DBU, lithium tert-butoxide or lithium diisopropylamide. The reaction mixture is stirred for about 8 hours to about 24 hours at room temperature, and the resulting imine is isolated by techniques well known in the art, such as extraction, to provide the imine of formula III.

In Scheme I, step C, the imine of formula III is alkylated with a compound of structure (2) wherein M+ is a suitable cation, such as lithium. Examples of compounds of structure (2) are methyllithium, butyllithium, and the like. For example, an excess of the compound of structure (2), such as methyl lithium in a suitable organic solvent, such as diethyl ether, is cooled to about −25° C. to about room temperature with about −10° C. being preferred. The imine of formula III in tetrahydrofuran is maintained at a temperature between about 5° C. and about 25° C., and added to the above cooled solution of compound (2). After about 20 minutes, the reaction is allowed to warm to room temperature. After stirring for about 1 to 2 hours at room temperature the crude compound of formula I is isolated by standard extractive techniques. For example, the reaction is diluted with water and the resulting layers are separated. The aqueous phase is extracted with diethyl ether or methyl tert-butyl ether, the organic layer and organic extracts are combined, washed with water, dried over anhydrous sodium sulfate, filtered, and concentrated under vacuum to provide the crude compound of formula I.

In Scheme I, step D, the crude compound of formula I may be contaminated with unreacted imine of formula III and non-alkylated compound of formula I. These impurities may be removed by chromatography on silica gel. The impurities are also readily removed by dissolving the crude compound of formula I in a suitable solvent, such as methanol and tetrahydrofuran or methyl tert-butyl ether, and treating the solution with about 1 equivalents of a suitable reducing agent for every mole of imine impurity present. Examples of suitable reducing agents are sodium borohydride, lithium aluminum hydride, and the like. Sodium borohydride is the preferred suitable reducing agent. The reaction is stirred for about 1 hour and about 0.1 equivalents to about 0.3 equivalents of a suitable acylating agent is added, such as a suitable anhydride or a suitable dicarbonate, with about 0.25 equivalents being preferred depending upon the amount of non-alkylated piperidine impurity present. Examples of suitable anhydrides are pivalic anhydride, acetic anhydride, propionic anhydride, and the like. Pivalic anhydride is the preferred suitable anhydride. Examples of suitable dicarbonates include di-tert-butyl dicarbonate, dimethyl dicarbonate, and the like. The reaction is then stirred for about 0.4 hours to about 2 hours, with about 1 hour being preferred. The purified compound of formula I is then isolated by acid-base extraction. For example, the mixture is partitioned between a suitable aqueous acid, such as 1 N aqueous HCl, and a suitable organic solvent, such as diethyl ether or methyl tert-butyl ether. The aqueous layer is washed with a suitable organic solvent, such as diethyl ether or methyl tert-butyl ether, and the aqueous is then made basic with a suitable base, such as 5 N sodium hydroxide. The aqueous is then extracted with a suitable organic solvent, such as diethyl ether or methyl tert-butyl ether. The combined organic extracts are washed with water, dried over anhydrous sodium sulfate, filtered, and concentrated under vacuum to provide the purified compound of formula I.

In addition, the compound of formula II can be dissolved in a suitable organic solvent, such as tetrahydrofuran and treated with a catalytic amount of a suitable base, such as potassium tert-butoxide or lithium diisopropylamide (LDA), and a compound of structure (2) as defined hereinabove. For example, an excess of the compound of structure (2) is added at a temperature between about 5° C. and 20° C. After about 20 minutes, the reaction is allowed to warm to room temperature. After stirring for about 18 hours at room temperature the crude compound of formula I is isolated by standard extractive techniques. For example, the reaction is diluted with water and the resulting layers are separated. The aqueous phase is extracted with diethyl ether or methyl tert-butyl ether, the organic layer and organic extracts are combined, washed with water, dried over anhydrous sodium sulfate, filtered, and concentrated under vacuum to provide the crude compound of formula I. Thus, by combining steps B and C, as described directly above, the compound of formula I can be prepared in one pot.

The following examples are illustrative only and represent typical syntheses of the compounds of formula I as described generally above. The reagents and starting materials are readily available to one of ordinary skill in the art. As used herein, the following terms have the meanings indicated: “eq” or “equiv.” refers to equivalents; “g” refers to grams; “mg” refers to milligrams; “L” refers to liters; “mL” refers to milliliters; “μL” refers to microliters; “mol” refers to moles; “mmol” refers to millimoles; “psi” refers to pounds per square inch; “min” refers to minutes; “h” refers to hours; “° C.” refers to degrees Celsius; “TLC” refers to thin layer chromatography; “HPLC” refers to high performance liquid chromatography; “R_{f}” refers to retention factor; “Rₜ” refers to retention time; “δ” refers to parts per million down-field from tetramethylsilane; “THF” refers to tetrahydrofuran; “DMF” refers to N,N-dimethylformamide; “DMSO” refers to methyl sulfoxide; “LDA” refers to lithium diisopropylamide; “aq” refers to aqueous; “EtOAc” refers to ethyl acetate; “iPrOAc” refers to isopropyl acetate; “MeOH” refers to methanol; “MTBE” refers to methyl tert-butyl ether, and “RT” refers to room temperature.

### Preparation 1

Preparation of 5-Bromo-3-methyl-1-trimethylsilylbenzo[b]thiophene.

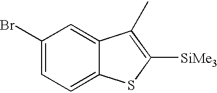

A solution of 5-bromo-3-methylbenzo[b]thiophene (149.1 g, 0.66 mole) in THF (1.4 L) under nitrogen was cooled to −78° C. and trimethylsilyl chloride (163 mL, 1.3 mole, 2 eq) was added dropwise. Lithium diisopropylamide (625 mL, 1.2 mole, 2 eq, 2.0 M solution in THF, heptane, ethylbenzene) was added and the mixture was stirred for 4 h. The solution was poured into a mixture of methyl tert-butyl ether and H₂O (3 L each). The layers were separated and the organic layer was extracted with 1 N HCl (2 L), then H₂O (2 L) and dried (Na₂SO₄). The solvent was removed by rotary evaporation to afford 237.3 g of crude product. The crude material was slurried in EtOH (400 mL) to afford 5-bromo-3-methyl-1-trimethylsilylbenzo[b]thiophene as a white granular solid (152.7 g, 78%, 3 crops). mp 64-67° C. IR (KBr) 1252, 1245, 841 cm⁻¹;

¹H NMR (300 MHz, CDCl₃) δ7.851 (d, 1, J=1.8 Hz), 7.69 (d, 1, J=8.5 Hz), 7.41 (dd, 1, J=8.5, 1.8 Hz), 2.48 (s, 3,), 0.42 (d, 9, J=3.4 Hz). ¹³C NMR (75 MHz, CDCl₃) δ143.6, 141.3, 137.9, 132.2, 126.9, 124.4, 123.4, 117.8, 14.4, 0.14. MS (FD) m/z 298 (M+). Anal. Calcd for C₁₀H₁₅BrSSi: C, 48.16; H, 5.05. Found: C, 48.19; H, 4.98.

### Preparation 2

Preparation of 1-(t-Butyloxycarbonyl)-4-(3-methylbenzo[b]thiophen-5-yl)-piperidin-4-ol.

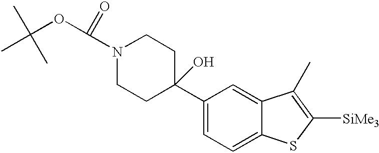

To a solution of 5-bromo-3-methyl-1-trimethylsilylbenzo[b]thiophene (211.7 g, 707 mmol) in THF (1 L) cooled to −78° C. under nitrogen was added n-BuLi (311 mL, 2.5 M solution in hexanes, 778 mmol) dropwise. After 30 min, N-Boc-piperidone (155.1 g, 778 mmol) in THF (816 mL) was added. After 2 h, the mixture was poured into H₂O and methyl tert-butyl ether (2 L each). The layers were separated and the organic layer was washed with 1N HCl (2.1 L), then H₂O (2.1 L) and dried (Na₂SO₄). The solvent was removed with a rotary evaporator to afford 348 g of crude 1-(t-butyloxycarbonyl)-4-(3-methyl-1-trimethylsilylbenzo[b]thiophen-5-yl)-piperidin-4-ol. Hexane (700 mL) was added to the crude product. After stirring overnight, the precipitate was filtered, washed with hexane, and dried in a vacuum oven for 2 h to give 246.3 g (83%) of 1-(t-butyloxycarbonyl)-4-(3-methyl-1-trimethylsilylbenzo[b]thiophen-5-yl)-piperidin-4-ol as a white powder. mp 141-145° C. IR (CHCl₃) 3595, 1680 cm⁻¹.

¹H NMR (300 MHz, CDCl₃) δ7.83 (d, 1, J=8.0 Hz), 7.81 (s, 1), 7.43 (dd, 1, J=8.2, 1.8 Hz), 4.06 (br s, 2), 3.28 (t, 2, J=12.2 Hz), 2.51 (s, 3), 2.09 (br s, 2), 1.79 (d, 2, J=12.2 Hz), 1.70 (s, 1), 1.49 (s, 9), 0.40 (s, 9). ¹³C NMR (75 MHz, DMSO) δ154.2, 145.9, 141.5, 140.3, 139.1, 134.1, 122.2, 121.8, 117.7, 78.6, 70.2, 38.0, 28.3, 14.4, 0.00. MS (FD) m/z 418 (M-1). Anal. Calcd for C₂₂H₃₃NO₃SSi: C, 62.97; H, 7.93; N, 3.34. Found: C, 63.28; H, 8.04; N, 3.44.

### Preparation 3

Preparation of N-t-Butoxycarbonyl-4-hydroxy-4-(6-methylbenzo[b]thiophen-2-yl)piperidine.

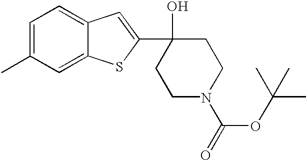

To a solution of 6-methylbenzo[b]thiophene (1.25 g, 8.43 mmol) in dry THF (20 mL) at −78° C. was added 1.6 M n-BuLi in hexanes (6.32 mL, 10.1 mmol). The solution was stirred at −78° C. for 40 min. 1-t-Butoxycarbonyl-4-piperidone (1.84 g, 9.27 mmol) dissolved in THF (10 mL) was added via a cannula at −78° C. The reaction mixture was stirred at −78° C. for 3 h. The reaction was then quenched with 50 mL of water. The mixture was extracted (3×75 mL) with EtOAc. The combined organic layers were dried over MgSO₄ and filtered. The filtrate was concentrated to an oil and allowed to stand 3 days in which time the material crystallized. The crystals were rinsed with a mixture of EtOAc/hexanes to give the intermediate title compound as yellow crystals (2.13 g, 72.6%). IR (KBr) 1681, 1429, 1246 cm⁻¹. FD+ MS 347.0 (M).

### Preparation 4

Preparation of N-t-Butoxycarbonyl-4-(8-methoxynaphth-2-yl)-4-piperidinol.

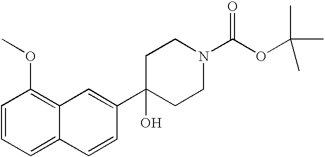

To a solution of 7-bromo-1-methoxynaphthalene (1.50 g, 6.33 mmol) in dry THF (30 mL) at −78° C. was added 1.6 M n-BuLi in hexanes (4.35 mL, 6.96 mmol). The solution was stirred at −78° C. for 15 min. N-t-Butoxycarbonyl-4-piperidone (1.51 g, 7.59 mmol) dissolved in THF (10 mL) was added via a cannula at −78° C. The reaction mixture was stirred at −78° C. for 2.5 h. The reaction was then quenched with 30 mL of saturated aqueous NH₄Cl solution. The mixture was extracted (2×150 mL) with EtOAc. The combined organic layers were then dried over MgSO₄ and filtered. The filtrate was concentrated and purified by silica gel chromatography (25% EtOAc/hexanes) to give the intermediate title compound as a white foam (1.42 g, 63%). IR (CHCl₃) 3350 (br), 1681 cm⁻¹. Ion Spray MS 358 (M+H)⁺; 240 (M-117(-(Boc+H₂O)))⁺; 430 (M+CH₃COO⁻)⁻. ¹HNMR (CDCl₃) δ8.31 (d, J=2.0 Hz, 1H), 7.79 (d, J=8.3 Hz, 1H), 7.60 (dd, J=8.8, 2.0 Hz, 1H), 7.34-7.40 (m, 2H), 6.81 (dd, J=7.1, 2.0 Hz, 1H), 4.03-4.06 (br m, 2H), 3.99 (s, 3H), 3.29 (br dt, J=13.0, 2.4 Hz, 2H), 2.12 (dt, J=13.0, 4.9 Hz, 2H), 1.79-1.83 (br m, 2H), 1.61 (br s, 1H), 1.48 (s, 9H).

### Preparation 5

Preparation of 4-(3-Methylbenzo[b]thiophen-5-yl)-piperidine Hydrochloride.

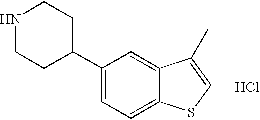

To a solution of 1-(t-butyloxycarbonyl)-4-(3-methyl-1-trimethylsilylbenzo[b]thiophen-5-yl)-piperidin-4-ol (458 g, 1.09 mol, from preparation 2) in CH₂Cl₂ (4.6 L) was added 871 mL (5.46 mol, 5.0 equiv) of triethylsilane. The mixture was cooled to −30° C. and 420 mL of trifluoroacetic acid (5.45 mol, 5.0 equiv) was added dropwise to the solution over 35 minutes. The mixture was stirred for 2.5 hours while gradually warming to 13° C. An additional 420 mL of trifluoroacetic acid was added over 15 minutes. After warming to room temperature over 3.5 hours, ice (6 L), water (5 L), and concentrated aqueous NaOH (628 mL, 12.0 mol, 11.0 eq) were added. The layers were separated and the aqueous layer was extracted with two 1.5 L portions of CH₂Cl₂. The organic layers were combined, dried (Na₂SO₄), and concentrated under vacuum to give a clear, colorless oil, which was redissolved in 4 L of ether. The hydrochloride salt was formed by dropwise addition of a solution of HCl in EtOAc (245 mL) until the slurry pH measured 2-3. The resulting slurry was stirred for 2 hours, filtered, rinsed with ether, and dried overnight in a vacuum oven at 45° C. to give 271 g of white crystalline 4-(3-methylbenzo[b]thiophen-5-yl)-piperidine hydrochloride (92.8% yield).

¹H NMR (500 MHz, DMSO) δ2.10-2.20 (m, 2), 2.30 (q, 2), 2.42 (s, 3), 2.93 (m, 1), 3.0-3.10 (m, 2), 7.09 (s, 1), 7.25 (d, 1), 7.57 (s, 1), 7.80 (d, 1); ¹³C NMR (75 MHz, DMSO) δ13.5, 29.6, 38.9, 43.4, 119.3, 122.7, 122.9, 123.2, 131.5, 137.7, 139.6, 140.9. Anal. Calcd for C₁₄H₁₈CINS: C, 62.79; H, 6.77; N, 5.23. Found: C, 62.66; H, 6.65; N, 5.24.

### Preparation 6

Preparation of 4-(6-methylbenzo[b]thiophen-2-yl)piperidine HCl.

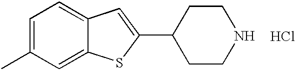

The title compound is prepared from N-t-butoxycarbonyl-4-hydroxy-4-(6-methylbenzo[b]thiophen-2-yl)piperidine (prepared in preparation 3) in a manner analogous to the procedure described in preparation 5.

### Preparation 7

Preparation of 4-(8-methoxynaphth-2-yl)piperidine HCl.

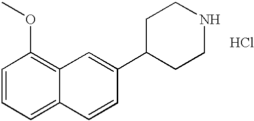

The title compound is prepared from N-t-butoxycarbonyl-4-(8-methoxynaphth-2-yl)-4-piperidinol (prepared in preparation 4) in a manner analogous to the procedure described in Preparation 5.

### EXAMPLE 1

Preparation of trans-2-Methyl-4-(3-methylbenzo[b]thiophen-5-yl)piperidine.

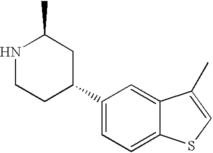

Preparation of N-Chloro-4-(3-methylbenzo[b]thiophen-5-yl)-piperidine.

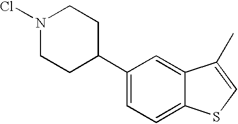

Scheme I, step A: To 10 g (37 mmol) of 4-(3-methylbenzo[b]thiophen-5-yl)-piperidine hydrochloride was added 160 mL of ether, 34 mL of H₂O and 41 mL (41 mmol, 1.1 equiv, prepared in preparation 5) of 1 M NaOH. The mixture was stirred until the solid dissolved and the layers were separated using a separatory funnel. The aqueous layer was extracted with 100 mL of ether and the combined organic layers were dried (Na₂SO₄) and evaporated to afford 8.73 g of 4-(3-methylbenzo[b]thiophen-5-yl)-piperidine. 4-(3-methylbenzo[b]thiophen-5-yl)-piperidine was dissolved in 83 mL of ether and 83 mL of THF, and 4.99 g (37 mmol, 1 equiv) of N-chlorosuccinimide was added. After stirring overnight, 100 mL of saturated aqueous NaHCO₃ was added and the mixture was transferred to a separatory funnel containing 100 mL of H₂O and 60 mL of ether. The layers were separated and the aqueous layer was washed with 10 mL of ether. The combined organic layers were dried (Na₂SO₄) and evaporated to afford 10.1 g (100% yield) of N-chloro-4-(3-methylbenzo[b]thiophen-5-yl)-piperidine; mp 57-61.5° C. IR (CHCl₃) 3009, 1602, 1471, 1448 cm⁻¹.

¹H NMR (300 MHZ, CDCl₃) δ7.78 (d, 1, J=8.2 Hz), 7.53 (d, 1, J=1.5 Hz), 7.21 (dd, 1, J=8, 2 Hz), 7.07 (d, 1, J=0.9 Hz), 3.6 (d, 2, J=11 Hz) 3.05 (t, J=12), Hz), 2.85-2.70 (m, 1), 2.43 (d, 3, J=1.2 Hz), 2.20-2.00 (m, 2, CH₂), 1.9 (br d, 2). ¹³C NMR (75 MHz, CDCl₃) δ141.0, 139.6, 137.5, 131.5, 123.4, 122.9, 122.7, 119.3, 43.6, 40.1, 29.7, 13.5. MS (FD) m/z 266 (M+). Anal. Calcd for C₁₄H₁₆CINS: C, 63.26; H, 6.07; N, 5.27. Found: C, 63.34; H, 6.06; N, 5.30.

Scheme I, step B: To 13.9 g (51.9 mmol) of N-chloro-4-(3-methylbenzo[b]thiophen-5-yl)-piperidine was added 207 mL of THF, 1.0 g (3.8 mmol, 0.073 equiv) of 18-crown-6 and a slurry of 10.16 g (assume 85% KOH and 15% H₂O, 156 mmol, 3 equiv) of potassium hydroxide in 4 mL of H₂O. After stirring for 16 h, the resulting solution of imine was dried over Na₂SO₄, filtered and the cake rinsed with 20 mL of THF.

Preparation of Final Title Compound.

Scheme I, step C: A solution of methyllithium in ether (185 mL, 1.5 M, 260 mmol, 5 equiv) was cooled to −10° C. and the precooled (5° C.) imine solution from above was added over 1 min. After 20 min, the cooling bath was removed and the mixture was allowed to stir at ambient temperature. After 2 h, 200 mL of H₂O was added, the layers were separated and the aqueous layer was extracted with ether (200 mL). The combined organic layers were washed with water (300 mL) and dried (Na₂SO₄). The solvent was evaporated to afford the crude final title compound, trans-2-methyl-4-(3-methylbenzo[b]-thiophen-5-yl)piperidine, (13.6 g) of a viscous oil.

Scheme I, step D: The crude final title compound was dissolved in 80 mL of methanol and 25 mL of THF and 0.55 g (12 mmol) of NaBH₄ was added. After 2 h, pivalic anhydride (2.8 g, 12 mmol), was added. After 2 h, the methanol was removed by evaporation and the mixture was partitioned between 200 mL of 1 N HCl and 100 mL of ether. The layers were separated and the aqueous layer was washed with ether. The aqueous layer was made basic with 5 N NaOH and extracted with ether (2×150 mL). The organic layers were dried and evaporated to afford 8.9 g (63% yield) of purified trans-2-methyl-4-(3-methylbenzo[b]-thiophen-5-yl)piperidine as a viscous oil.

### EXAMPLE 2

Preparation of trans-2-Methyl-4-(6-methylbenzo[b]thiophen-2-yl)piperidine.

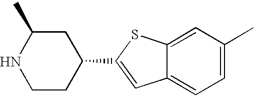

Scheme I, steps A-D: The title compound is prepared in a manner analogous to the procedures described in example 1 from 4-(6-methylbenzo[b]thiophen-2-yl)piperidine HCl prepared in preparation 6.

### EXAMPLE 3

Preparation of trans-2-Methyl-4-(8-methoxynaphth-2-yl)piperidine.

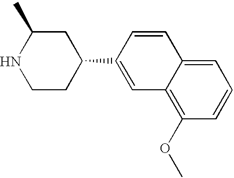

Scheme I, steps A-D: The title compound is prepared in a manner analogous to the procedures described in example 1 from 4-(8-methoxynaphth-2-yl)piperidine HCl prepared in preparation 7.

### EXAMPLE 4

Preparation of trans-2-Methyl-4-(2-naphthyl)piperidine.

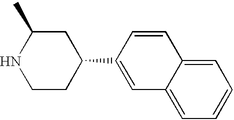

Scheme I, steps A-D: The title compound was prepared in a manner analogous to the procedures described in example 1 from 4-(2-naphthyl)piperidine HCl.

### EXAMPLE 5

Preparation of trans-2-Methyl-4-(4-methylphenyl)piperidine.

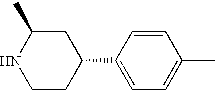

Scheme I, steps A-D: The title compound was prepared in a manner analogous to the procedures described in example 1 from 4-(4-methylphenyl)piperidine HCl.

### EXAMPLE 6

Preparation of trans-2-Methyl-4-(3-methoxyphenyl)piperidine.

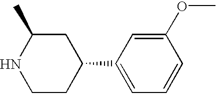

Scheme I, steps A-D: The title compound was prepared in a manner analogous to the procedures described in example 1 from 4-(3-methoxyphenyl)piperidine HCl.

### EXAMPLE 7

Preparation of trans-2-Methyl-4-(4-methoxybenzo[b]thiophen-2-yl)piperidine.

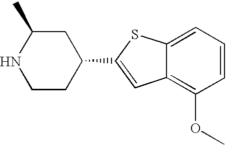

Scheme I, steps A-D: The title compound was prepared in a manner analogous to the procedures described in example 1 from 4-(4-methoxybenzo[b]thiophen-2-yl)piperidine HCl.

We claim:

1. A process for preparing a compound of formula I: 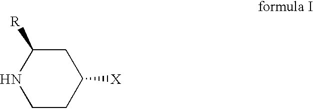
wherein
R represents C₁-C₄ alkyl; and
X represents an alkyl, alkenyl, cycloalkyl, aryl, substituted aryl, heterocycle, or substituted heterocycle, comprising treating a compound of formula II: 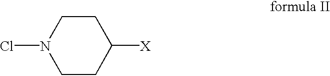
with a suitable crown ether and a suitable base followed by addition of a compound of formula R⁻M⁺ wherein M⁺ is a suitable cation.

2. The process according to claim 1 wherein R represents methyl.

3. The process according to any one of claims 1 or 2 wherein the suitable cation is Li⁺.

4. The process according to any one of claims 1 to 3 wherein the suitable crown ether is 18-crown-6 and the suitable base is potassium hydroxide.

5. The process according to claim 1, further comprising purifying the compound of formula I by treatment with a suitable reducing agent followed by addition of a suitable acylating agent and then acid-base extraction of the mixture.

6. The process according to claim 5 wherein the suitable acylating agent a suitable anhydride.

7. The process according to any one of claims 5 or 6 wherein the suitable anhydride is pivalic anhydride.

8. The process according to any one of claims 5 to 7 wherein the suitable reducing agent is sodium borohydride.

9. The process according to claim 4, further comprising purifying the compound of formula I by treatment with a suitable acylating agent.

10. The process according to claim 9 wherein the suitable acylating agent is a suitable anhydride.

11. The process according to any one of claims 9 or 10 wherein the suitable anhydride is pivalic anhydride.

12. The process according to any one of claims 9 to 11 wherein the suitable reducing agent is sodium borohydride.

13. The process according to any one of claims 9 to 12 wherein the suitable crown ether is 18-crown-6 and the suitable base is potassium hydroxide.

14. The process according to any one of claims 9 to 13 wherein the acid-base extraction comprises partitioning the mixture between a suitable aqueous acid and a suitable organic solvent; separating the layers; washing the aqueous layer with a suitable organic solvent; making the aqueous layer basic with a suitable base; and extracting the aqueous layer with a suitable organic solvent.

15. A compound of the formula I: 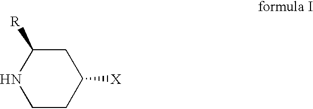
wherein
R represents C₁-C₄ alkyl; and
X represents an alkyl, alkenyl, cycloalkyl, aryl, substituted aryl, heterocycle, or substituted heterocycle.

16. A compound according to claim 15 wherein R is methyl.

17. A compound according to any one of claims 15 or 16 wherein X is heterocycle or substituted heterocycle.

18. A compound according to any one of claims 15 or 16 wherein X is aryl or substituted aryl.

19. A compound according to any one of claims 15 or 16 wherein X is benzothiophene or substituted benzothiophene.

20. A compound according to claim 19 wherein the benzothiophene is substituted with methyl.

21. A compound which is trans-2-Methyl-4-(3-methylbenzo[b]-thiophen-5-yl)piperidine.

22. A compound according to any one of claims 15 or 16 wherein X is benzofuran or substituted benzofuran.

23. A compound according to any one of claims 15 or 16 wherein X is indole or substituted indole.

24. A process for preparing a compound of formula I: 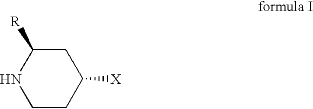
wherein
R represents C₁-C₄ alkyl; and
X represents an alkyl, alkenyl, cycloalkyl, aryl, substituted aryl, heterocycle, or substituted heterocycle, comprising treating a compound of formula II: 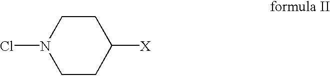
with a suitable base followed by addition of a compound of formula R⁻M⁺ wherein M⁺ is a suitable cation.

25. The process according to claim 24 wherein R represents methyl.

26. The process according to any one of claims 24 or 25 wherein the suitable cation is Li⁺.

27. The process according to any one of claims 24 to 26, further comprising purifying the compound of formula I by treatment with a suitable reducing agent followed by addition of a suitable acylating agent and then acid-base extraction of the mixture.

28. The process according to any one of claims 24 to 27 wherein the suitable acylating agent a suitable anhydride.

29. The process according to any one of claims 24 to 28 wherein the suitable anhydride is pivalic anhydride.

30. The process according to any one of claims 24 to 29 wherein the suitable reducing agent is sodium borohydride.
